(19) United States
(12) Patent Application Publication
(10) Pub. No.: US 2026/0038633 A1
(43) Pub. Date: Feb. 5, 2026

(54) Systems and methods for performing Shared Match Differentiation

(71) Applicant: Arun Christopher Konanur, London (CA)

(72) Inventor: Arun Christopher Konanur, London (CA)

(21) Appl. No.: 18/793,774

(22) Filed: Aug. 3, 2024

(51) Int. Cl. G16B 20/40; G16B 40/20

(52) U.S. Cl. CPC G16B 20/40; G16B 40/20

(57) ABSTRACT

A bioinformatic system that differentiates collections of shared autosomal DNA (atDNA) matches is disclosed. The invention consists of two main parts: a process which can differentiate “Favourable” Trios of individuals from “Unfavourable” Trios, whilst doing so in a computationally efficient manner, compatible with real-time reporting of DNA testing results; and a desktop/spreadsheet prototype which performs a bioinformatic assessment of three individuals using the matches from their DNA test results by utilizing the aforementioned process. The process may also be applied to assess larger (n-element) collections of individuals, to verify the integrity of datasets produced by other bioinformatic processes, to validate collections of DNA matches used as inputs to bioinformatic processes, and to assess the integrity of ancestral lines connecting individuals of unknown or uncertain pedigree within the context of established family groupings.

### FIELD OF THE INVENTION

The present invention relates to a system that performs Shared Match Differentiation (SMD), a method of evaluating sets of DNA matches to determine whether they originate from a Common Ancestor (“Favorable” collections) or from divergent ancestral lines (“Unfavorable” collections).

### BACKGROUND OF THE INVENTION

Direct-to-consumer autosomal DNA (atDNA) testing for the purpose of ancestry analysis was introduced in 2007, and since then millions of consumers have purchased test kits from one or more commercial entities which offer this service (23andMe, AncestryDNA, Family Tree DNA, MyHeritage, etc.). In each case, an individual Testor's atDNA is sampled along roughly 700,000 single-nucleotide polymorphisms (SNPs), which are in turn compared against the test results of other customers of that same service (as many as 25 million other tests depending on the service), in order to generate a list of member matches—generally presented as a roster of member names and/or test kit numbers ranked by the amount of DNA shared with the test taker. This list of member matches may consist of anywhere from several hundred names/subject identifiers to more than 100,000 such matches, depending on the prevalence of genetically related subjects already tested, and the degree of endogamy present in the Testor's ancestral or ethnic subgroup.

Beyond a ranked listing of the Testor's autosomal DNA matches, most commercial testing services provide detailed information on individual DNA matches via a linked roster of individuals whose DNA test kit matches both the Testor and the selected DNA match (henceforth, the “Match”). These are known as “Shared Matches” and can be of great utility in identifying the common ancestral origins of the Testor, the Match, and the Shared Match.

However within any such “Trio” (Testor, Match, and Shared Match) there remains a non-zero probability that the DNA connections among the Trio arise not from an individual instance of Identical by Descent DNA segments, suggesting a single Common Ancestor (a “Favorable” Trio), but rather from the confluence of multiple ancestral lines, each line connecting two elements of the Trio (an “Unfavorable” Trio).

Without a reliable method for differentiating Favorable versus Unfavorable Trios, commercial testing services are left with the uncertain proposition of either reporting inaccurate or misleading data, or withholding data on Shared Matches, despite its usefulness. Inasmuch as the probability of Unfavorable associations varies inversely with the DNA linkage shared by the elements of the Trio, an intermediary approach, employed by some testing services, has been to report Shared Matches above a certain threshold—a well-known testing service uses 20 centiMorgans for this purpose—and suppressing reports of Shared Matches which fall below this threshold. However, an invidious distinction such as this, by its very nature, does nothing to ensure that data above the threshold is of uniform quality, and accepts the possibility that useful Trios have been suppressed.

Further, recent developments in the marketplace have led some commercial testing services to provide unrestricted reporting of Shared Matches to users on a subscription basis, increasing the need for a reliable protocol for differentiating Favorable from Unfavorable Trios. As a practical matter, any such differentiating protocol should be of sufficient computational efficiency as so to be deployed in a manner where shared match Trios can be filtered in real-time, to conform with established reporting practices.

Beyond this immediate and pressing need, the emergence of bioinformatic expert systems as a method of analyzing problems in genetic genealogy via Artificial Intelligence and its associated schema gives rise to the need for independent verification of both the output and source materials of such processes. Establishing the common ancestral origins of datasets provides valuable insights and can function as a “proofreading” of genealogical bioinformatic output.

### SUMMARY OF THE INVENTION

This invention is directed to differentiate collections of shared DNA matches (typically 3-element trios and their compounds) by using Axiomatic Set Theory to facilitate the bioinformatic evaluation of their ancestral origins. The most basic such collection is the “Trio” consisting of: a Testor; a second subject, taken from the Testor's roster of DNA matches (the “Match”); and a third subject (the “Shared Match”) who appears on the match rosters of both the Testor and the Match.

Although such shared match “Trios” are germane to the shared DNA match reporting of genealogical DNA testing services, n-element collections of shared DNA matches occur elsewhere in bioinformatic processes and analyses, and so the ability to verify the integrity of a dataset—either as the product of a bioinformatic process, or prior to the utilization of a given dataset as input in other bioinformatic operations—is of the utmost utility. The validity of larger collections of shared matches may be inferred through the validation of overlapping subsets of the greater collection.

As a lightweight computational protocol SMD is well suited to deployment in real-time reporting systems, where lists of shared matches are assembled on demand from a server backend. The ability to filter out Unfavorable Trios—collections of shared DNA matches which do not originate from a single common ancestor—increases the utility of shared match reporting, as users are confident that the Shared Match trios they receive are of genealogical significance.

Lastly, the validation of shared matches of individuals from a given ancestral line against collections of shared matches from an established pedigree may suggest or refute the presence or absence of non-paternity-events (NPEs), inasmuch as ambiguity may otherwise arise in shared matches from distant genealogical relations. Understanding whether these distant matches form Favorable or Unfavorable collections is an important factor in drawing larger conclusions.

### BRIEF DESCRIPTION OF THE DRAWINGS

In order to facilitate a fuller understanding of the present invention, reference is now made to the accompanying drawings. These drawings should not be construed as limiting the present disclosure, but are intended to be exemplary only.

FIG. 1 illustrates a real-world example of a Favorable Trio and its accompanying inheritance diagram.

FIG. 2 illustrates a real-world example of an Unfavorable Trio and its accompanying inheritance diagram.

FIG. 3 illustrates the multitude of possible genealogical connections to a Favorable Trio.

FIG. 4 illustrates the limited number of possible genealogical connections to an Unfavorable Trio.

FIG. 5 illustrates that the conditions through which an individual might match the members of an Unfavorable trio are all instantiations of the same basic scenario.

FIG. 6 presents a table of potential criteria employed by SMD to differentiate Favorable and Unfavorable Trios.

FIG. 7 illustrates that the two types of Trios share an underlying common structure—a Favorable Dyad—and that the matches common to this dyad are in turn reduced in number when a third element is added to form a Trio.

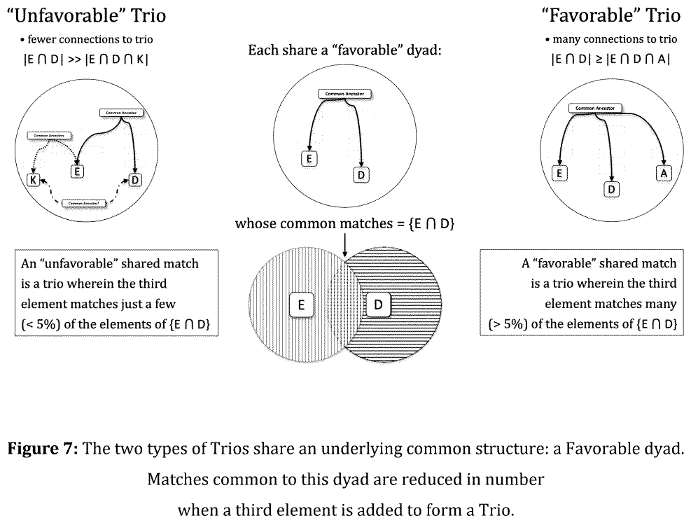

FIG. 8 presents the ten instantiations of the Differentiation Value V required to evaluate a collection of five individuals.

FIG. 9 is a process flowchart illustrating Shared Match Differentiation from the perspective of the end user of the SMD Utility.

FIG. 10 illustrates the user interface layout of the SMD Utility.

FIG. 11 illustrates the data entry process of the SMD Utility.

FIG. 12 is a process flowchart illustrating how SMD may be deployed to facilitate real-time differentiation of Shared Matches within the backend reporting of a commercial provider of genealogical DNA testing.

### DETAILED DESCRIPTION OF THE INVENTION

### I. The SMD Process

Favorable Vs. Unfavorable Collections of DNA Matches

Prior to delineating the bioinformatic considerations at the heart of Shared Match Differentiation (SMD), it may be useful to examine the types of collections SMD was created to differentiate.

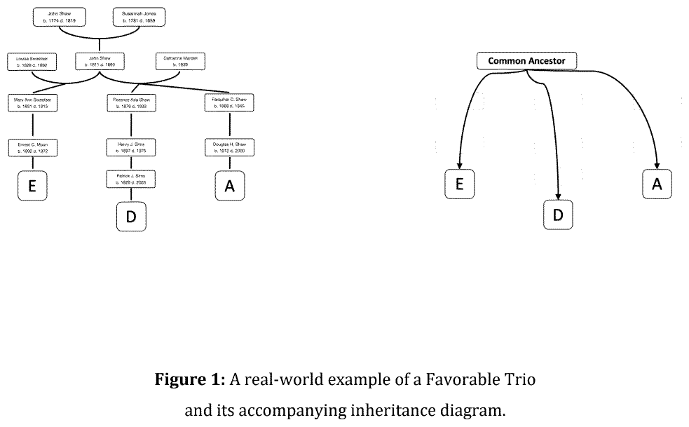

FIG. 1 illustrates a real-world example of three test subjects descended from a Common Ancestor and the reductive inheritance diagram drawn from these verified pedigrees. Inasmuch as the three elements of this collection share a common ancestor, genealogists regard this as a “Favorable” Trio-meaning that the DNA match shared by these individuals results from a single common convergence of their respective ancestral lines, such that the collection supports further research into this shared pedigree.

Such a convergence is typically observed in the form of a Most Recent Common Ancestral Couple (MRCAC). The convergence of FIG. 1's ancestral lines in the person of a single individual as extrapolated to an MRCAC by the fact that all individuals are the biological offspring of two parents.

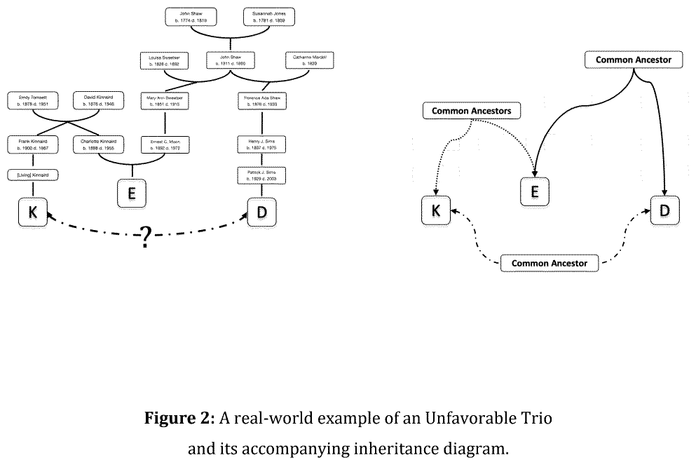

In contrast, FIG. 2 illustrates a real-world example of an “Unfavorable” Trio, where pairs of individuals within the Trio share a common ancestor, but the trio as whole is connected via separate, divergent ancestral lines. This arrangement is considered “Unfavorable” from a genealogical standpoint because the Trio does not support further insight into the pedigree of any of its elements and, in effect, asks more questions than it answers.

While genealogists have long recognized the importance of the preceding distinction, DNA testing services have lacked an efficient means of discriminating between Favorable and Unfavorable Trios—short of the generality that the likelihood of encountering an Unfavorable Trio tends to increase as the DNA shared amongst the elements of the Trio decreases.

However, recently offered subscription-based reporting packages from some commercial testing providers—offerings which report the entirety of a Testor's Shared Matches—have brought the necessity of a computationally lightweight differentiation protocol to the fore, increasing the utility of SMD.

### Bioinformatic Differentiation of Trios

Genealogical bioinformatics examines the genetic and genealogical properties of each type of collection—Favorable and Unfavorable—in order to arrive at a mathematical basis for the differentiation of the two types of Trios. In keeping with best bioinformatic practices, and in order to be useful in applications which require on-the-fly sorting, the mathematical basis for this differentiation should be as computationally lightweight and straightforward as possible.

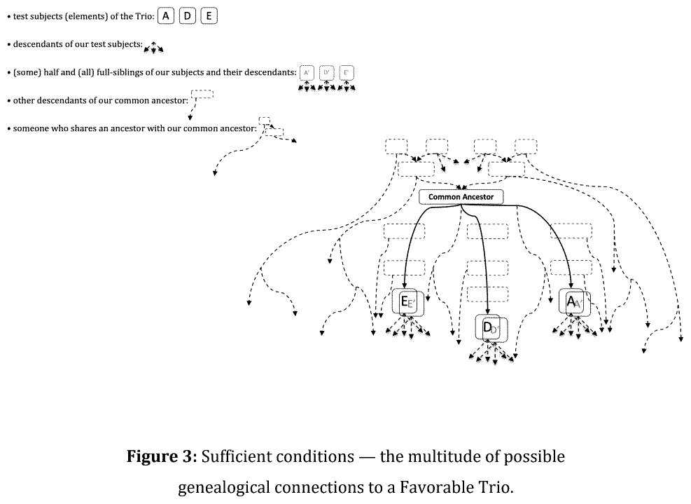

FIG. 3 presents the inheritance diagram of FIG. 1 amended to reflect the array of genealogical connections which might possibly satisfy the organizing principle of a three-element Favorable shared match: “Given that individuals A, D, and E share a single Common Ancestor (or Most Recent Common Ancestral Couple), what genealogical relations might possibly also match individuals A, D, and E?” The key to the left of FIG. 3 illustrates the multitude of possible genealogical solutions to this query.

Because the single Common Ancestor is shared by all three individuals in the Favorable Trio, only one condition (connection to the Common Ancestor) needs to be satisfied for a hypothetical individual to match every individual in the Trio. For this reason, genealogical connections to the Favorable Trio are an example of “sufficient conditions”—a single criterion which may be satisfied in any genealogically possible manner for an individual to match the elements of our Favorable Trio.

In addition to the number of possible connections to our three subjects, the variety of genealogical relationships amongst these connections suggests that the shared linkage between our Trio and these DNA matches will necessarily range from the significant linkage of direct descendants and various flavors of siblings to the small segments shared by distant cousins.

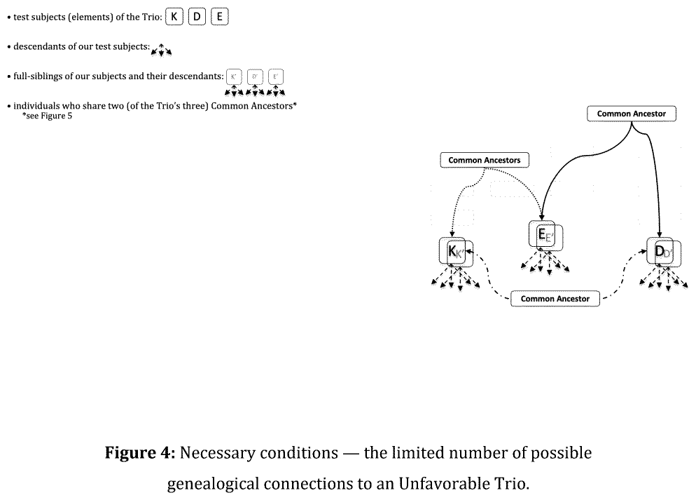

FIG. 4 presents the inheritance diagram of FIG. 2 amended to reflect the array of genealogical connections which might possibly satisfy the organizing principle of a three-element Unfavorable shared match.

Because the three Common Ancestors of the Unfavorable Trio are distributed across subsets of the Trio's elements, two conditions (connections to two of the three Common Ancestors) must be satisfied for a hypothetical individual to match the Unfavorable Trio's Subjects. For this reason, genealogical connections to the Unfavorable Trio are an example of “necessary conditions”-concurrent criteria which must be satisfied in order for an individual to match the elements of our Unfavorable Trio.

The necessity of maintaining genetic connections to two distinct ancestral lines (and thereby satisfying two necessary conditions) translates to fewer possible individuals matching the elements of an Unfavorable Trio. Direct descendants of the Unfavorable Trio's elements may qualify, as may full-siblings of our elements and their direct descendants.

The possibility (or impossibility) of further individuals matching all three elements of our Unfavorable Trio greatly depends on the relationship of our Trio's elements to the genealogical location where the Trio's ancestral lines converge/diverge.

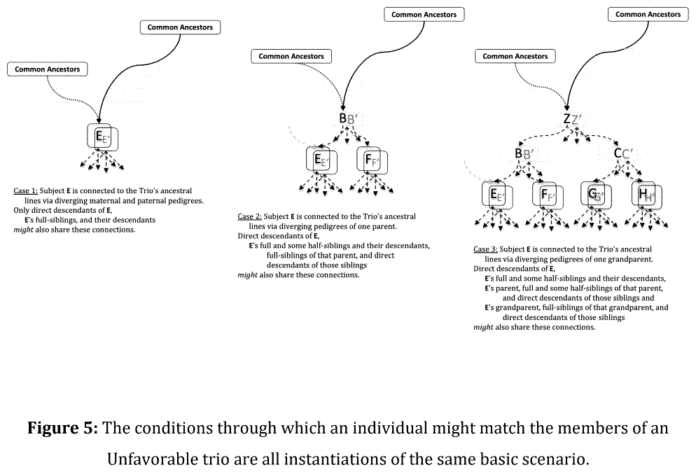

FIG. 5 presents three instances of the convergence of ancestral lines in the vicinity of our test subjects: in Case 1, shared ancestral lines converge at subject E's generation, limiting the individuals who match our Unfavorable trio's subjects to our test subject, descendants of the test subject, full-siblings of the subject, and the descendants of these full-siblings.

FIG. 5, Case 2 illustrates the convergence of shared ancestral lines in the generation preceding our test subject E and so—in addition to the individuals of Case 1—our possible connections include one of E's parents, E's parent's full-siblings, and descendants of those siblings, such as E's 1^{st} cousin F and their descendants.

FIG. 5, Case 3 illustrates the possible connections to our Unfavorable Trio when the convergence of common ancestral lines is two generations removed from subject E. From this case, it should be apparent that the generation of our subject E relative to the convergence of common ancestral lines does nothing to affect the number of possible connections to our Unfavorable Trio, as Cases 1, 2, and 3 are in reality the structure of Case 1 viewed from one and two generations removed.

As such, the number of individuals who might possibly match the three subjects of our Unfavorable Trio is necessarily restricted to reasonably close genealogical relations of any one of the subjects of our Trio, which in turn provides us with a further basis for the differentiation of individuals matching all three elements of our Trio. Inasmuch as many ancestral ethnicities originate from relatively closed populations, Unfavorable (and to a lesser extent, Favorable) Trios may also exhibit an abundance of matches that share small amounts of DNA with the Trio's three subjects.

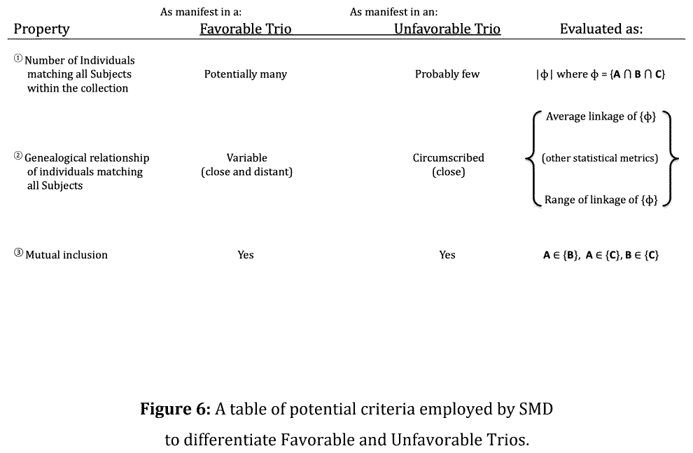

FIG. 6 summarizes the properties by which SMD might potentially differentiate Favorable and Unfavorable Trios of shared matches and the mathematical basis through which these properties might in turn be evaluated by SMD.

In order to accommodate the potential selection of individuals that don't form a Trio of any sort, FIG. 6, property \textcircled{3} adds the stipulation of mutual inclusion—that each element of the Trio is included amongst the DNA matches of the other elements. Since DNA matching is symmetric (i.e.: if A matches B, then B necessarily matches A), we only need to verify half of the Subject/Match permutations of any Trio.

The criteria enumerated in FIG. 6 are simple enough to be tabulated on the fly, to facilitate the real-time differentiation of Favorable and Unfavorable Trios within the reporting framework of commercial DNA testing services—which typically assemble rosters of shared DNA matches as and whenever requested. In practice, the statistical distribution of individuals matching our Trio is a greater function of which individuals elect to take a test, than of the properties of the Trio itself, so properties \textcircled{1} and \textcircled{3} are utilized almost exclusively by SMD in the evaluation of Trios of shared matches.

When considering whether a Trio has “many” or “few” individuals matching the Trio's three subjects, SMD evaluates the number of individuals that match all three subjects (|A∩B∩C|) and divides this number by the number of individuals which match the elements that form a Favorable dyad within the trio (for example, (|A∩B∩|, |B∩C|, or |A∩C|).

However, because the Favorable/Unfavorable status of the trio remains invariant, even as the elements of the Trio (Testor, Match, Shared Match) may switch positions, SMD evaluates the intersections of each of the Trio's dyads (|A∩B∩|, |A∩C|, |B∩C|) and prefers the dyad with largest intersection set.¹ ¹ An exception being when the elements of the preferred dyad share more than 1,400 cM of linkage—so as to avoid the liabilities of a Trio which forms a “two-legged stool,” with two elements disproportionately close from a genetic standpoint.

Therefore, SMD's Differentiation Value (V) obtained from the evaluation of an unknown Favorable/Unfavorable Trio is:

$$V = \frac{\vert A ⋂ B ⋂ C \vert}{{}_{max} ( \vert A ⋂ B \vert , \vert A ⋂ C \vert , \vert B ⋂ C \vert )}$$

As the value of the numerator in this fraction cannot exceed the value of the denominator—at best, their values could be equal—V is typically expressed as a percentage, which the SMD Utility's application log displays to two decimal places.

Additionally, because close genealogical relations of the Trio's subjects would be expected to match the elements of the Trio, whether Favorable or Unfavorable, SMD excludes from its intersection sets individuals sharing more than 650 centiMorgans of DNA with any Trio subject.

Similarly, because small DNA matches are notoriously unreliable, and because Trios exhibiting endogamy (a frequent aspect of Unfavorable collections) tend to produce a surfeit of small DNA matches, SMD also excludes individuals who share less than 20 centiMorgans of DNA with every element of the Trio from its intersection sets.

As such, the full formulation of V, SMD's Differentiation Value, for a Trio composed of individuals A, B, and C, becomes:

$$V = \frac{\vert A ⋂ B ⋂ {C}_{n} \vert}{{}_{max} ( \vert A ⋂ B \vert , \vert A ⋂ {C}_{n} \vert , \vert B ⋂ {C}_{n} \vert )} \vert {}_{max} L ( x , y ) \leq 650 cM AND {}_{max} L ( x , y ) \geq 20 cM$$

Where L is shared DNA linkage and x, y∈{A, B, C}

A Differentiation Value of 5% or less represents the typical discrimination point between Favorable and Unfavorable Trios. However, this threshold may be empirically fine-tuned through the use of AI-style “training” utilizing the datasets of a commercial provider of DNA tests.

### Differentiation of Larger Collections

SMD may be used to assess larger collections of individuals by evaluating 3-element subsets of the larger whole. For instance, a 4-element collection (A, B, C, D) would be evaluated as 4 Trios: (A, B, C), (A, B, D), (A, C, D), and (B, C, D). If the Differentiation Value (V) for every subset indicates that all are Favorable Trios, then the four individuals share a common ancestor.

On the other hand if, say, only subsets (A, B, C) and (B, C, D) form Favorable Trios, then it's possible that individuals A and D do not share sufficient (or any) linkage. One possible scenario would be that individuals B and C share a common ancestral couple, whilst individual A is related to B and C through one member of that couple whilst D is related to B and C through the other member of their common ancestral couple. It's important to note that these inferences are made possible whether or not family trees have been constructed for any of our four subjects, as SMD provides prospective guidance for genealogical research in addition to retrospective verification of a priori family trees.

All collections of individuals greater than three elements are evaluated as aggregations of 3-element subsets, each subset evaluated with a unique instantiation of the Differentiation Value formula V. The number of 3-element subsets(S) for a collection of n-elements is given by the formula:

$$S = \frac{n !}{( n - 3 ) ! 3 !}$$

And so the number of instantiations of V required to evaluate collections of up to ten individuals is:

| Number of | Number of |
|---|---|
| Individuals | Differentiation Values |
| 3 | 1 |
| 4 | 4 |
| 5 | 10 |
| 6 | 20 |
| 7 | 35 |
| 8 | 56 |
| 9 | 84 |
| 10 | 120 |

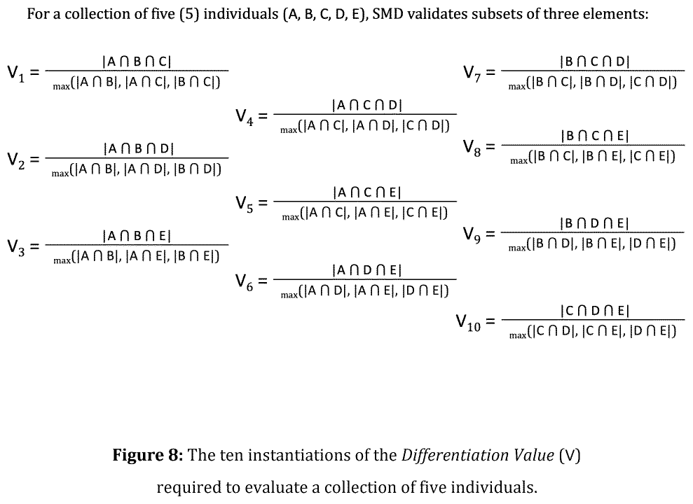

FIG. 8 presents the ten instantiations of V required to evaluate a collection of five individuals. Although the effort required to perform this work manually might appear onerous, the computational process of evaluating and tabulating the results of these formulae are greatly assisted by the SMD Utility: a scripted environment which runs in Microsoft Excel.

### II. SMD on the Desktop Computing Platform Via the SMD Utility

SMD's computationally lightweight protocols lend themselves to the evaluation of curated and arbitrary collections of individuals via the SMD Utility.

The SMD Utility takes as its input the full set of DNA matches for three individual Test Subjects (nominally the Testor, Match, and Shared Match) and evaluates these DNA matches per SMD's protocols, ultimately assigning the collection the status of NON-TRIO, FAVORABLE or UNFAVORABLE.

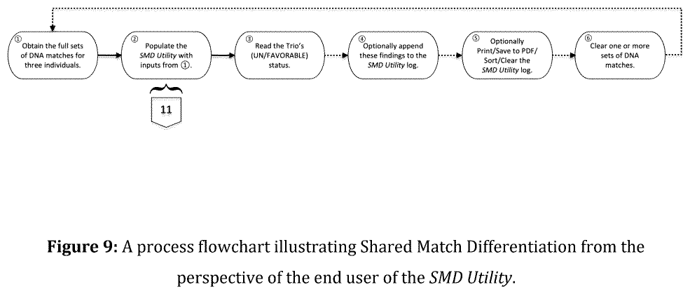

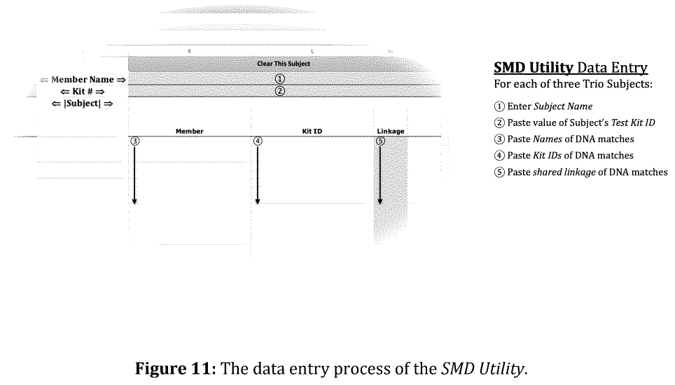

FIG. 9 presents a flowchart of the end user's experience of the SMD Utility. (Step (2) references the data entry methods illustrated in FIG. 11)

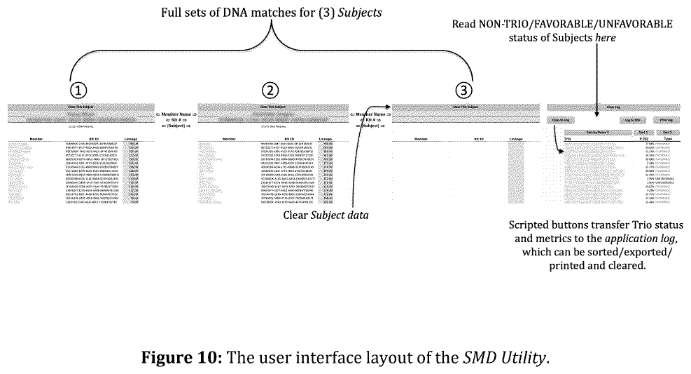

FIG. 10 illustrates the interface of the SMD Utility. The left portion of the interface has space for the particulars and DNA matches of three (3) Test Subjects, which are typically individuals identified by the Shared Match reporting of a commercial DNA testing service (however, sets of DNA matches from any three individuals may be used.) The data input areas can accommodate up to 150,000 DNA matches per Subject. Scripted buttons facilitate independently clearing out each subject's data, to more easily explore permutations of collections where some elements remain invariant.

The right portion of the SMD Utility displays the NON-TRIO/FAVORABLE/UNFAVORABLE status of the Test Subjects for quick and easy top-line evaluation, along with a scripted button that copies the metrics and status of the current Trio to the SMD Utility's application log. The application log can maintain an audit trail of mutually associated trios, so that collections of individuals with more than three elements may be analyzed.

The SMD Utility identifies each Trio through the concatenation of [Subject 1 name]/[Subject 2 name]/[Subject 3 name]. (Test Subject names have been obscured in FIG. 10 for privacy reasons, along with the names of individual DNA matches). In the event that the name of one or more Subjects is unavailable or otherwise redacted, the SMD Utility will substitute the Test Kit ID of that Subject in place of the Name.

The Differentiation Value for each Trio is calculated per the SMD protocols previously described, and the FAVORABLE/UNFAVORABLE designations are made using a 5% split point, which can be adjusted as and when the SMD protocols are trained using a commercial dataset. The NON-TRIO designation is applied to collections of individuals which fail to pass the mutual inclusion test of FIG. 6, property \textcircled{3}.

III. SMD with Real-Time Reporting and Other Bioinformatic Uses

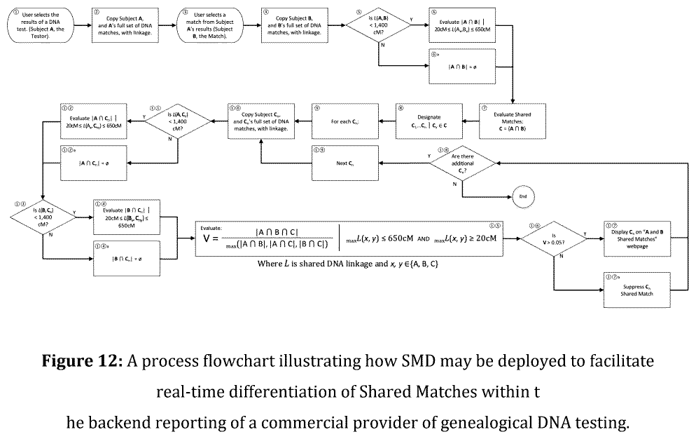

FIG. 12 presents a process flowchart illustrating how SMD may be deployed within the backend reporting of a commercial provider of genealogical DNA testing.

Given a test-taker (or Testor, Subject A) and a DNA Match (Subject B), standard practice is to build a list of shared matches on demand—typically by listing all DNA matches shared by Subjects A and B above an arbitrary reporting threshold (say, 20 cM). This practice potentially omits valuable Shared Matches below 20 cM and presumes that all Trios sharing more than 20 cM linkage are valid, Favorable Trios—which is by no means universally the case.

Deploying the process of FIG. 12 ensures that only quality data is transmitted to the end user; the computationally lightweight nature of the SMD process ensures that the identification of Favorable Trios can be performed with a minimal investment of computing resources.

Because all autosomal DNA matching exists in the same bioinformatic “space,” the SMD process may be utilized to validate or “proofread” collections of DNA matches produced by other bioinformatic processes, or employed as a pre-process in instances where a bioinformatic process accepts collections of DNA matches as its input.

One such process is CMA (Correlated Multiphasic Analysis, USPTO application Ser. No. #17/470,321), which uses the DNA Matches of a focal subject (or Nexus), and the DNA matches of known genealogical relations of that Nexus, to generate a collection of individuals related to the Nexus through the pedigree of an otherwise undocumented “brick wall” ancestor. Because CMA uses Set Theory to generate large collections of individuals throughout the entirety of a subject's DNA matches, there remains a possibility, however remote, that some subsets of these collections might contain “Unfavorable” Trios or larger n-element collections, and therefore represent spurious results.

SMD may also be deployed to filter out Unfavorable collections or otherwise “proofread” CMA's output. This is particularly useful when CMA-derived collections are utilized as an input for AASK (Axiomatic Ancestral Stratification by Kinship, USPTO application Ser. No. #18/641,045), a process which takes a roster of individuals—typically generated by CMA, though the entirety of a Subject's DNA matches may also be used—along with the full set of DNA matches of every individual in that roster, and organizes these individuals hierarchically, generation by generation, as they relate to the source individual who generated the elements of the roster. In such a GIGO (garbage in, garbage out) situation, the utility of verifying the integrity of a dataset prior to computationally-intensive processing cannot be underestimated.

1. A process for performing Shared Match Differentiation (SMD) of autosomal DNA (atDNA) matches, independent of any specific testing provider or tabulating mechanism.

2. The process of claim 1, where the full set of DNA matches of a “Trio” consisting of: a test subject (“Testor”); an individual selected from the Testor's roster of DNA matches (the “Match”); and an individual who appears on both the Testor's and the Match's roster of DNA matches (the “Shared Match”) are evaluated using the SMD protocol in order to determine whether they share common or divergent ancestral origins.

3. The process of claim 1, whereby the DNA matches of three individuals from an existing family line may be evaluated using the SMD protocol in order to determine whether they share common or divergent ancestral origins.

4. The process of claim 1, whereby the magnitude of the set-theoretic intersection of three sets of DNA matches is further reduced through the tabulation of the amount of DNA each element of the intersection set shares with the Test Subjects themselves.

5. The process of claim 1, whereby the set-theoretic intersection of the DNA matches of a Trio's constituent dyads is evaluated, with each intersection set further reduced through the tabulation of the amount of DNA each element of the intersection set shares with the Test Subjects of their respective dyad.

6. The process of claim 1, whereby the set-theoretic intersection of the DNA matches of a Trio's constituent dyads is discarded (set to null) if the elements of the dyad share more than 1,400 cM of linkage.

7. The process of claim 1, whereby the set-theoretic intersection of the DNA matches of the Trio's constituent dyads with the largest magnitude is preferred.

8. The process of claim 1, whereby a differentiation value is obtained by dividing the magnitude of the intersection set of the Trio by the magnitude of the intersection set of preferred dyad.

9. The process of claim 1, whereby an initial differentiation value threshold of 5% is used to differentiate between “Favorable” and “Unfavorable” Trios.

10. The process of claim 1, whereby the differentiation value threshold may be further adjusted and refined by training the SMD model on a large dataset, such as those typically accessible through commercial genealogical DNA testing providers.

11. The process of claim 1, whereby collections of individuals which do not mutually include each other amongst their DNA matches are labelled as “non-trios”.

12. The process of claim 1, programmed to run on the desktop platform as the SMD Utility, a scripted environment in Microsoft Excel.

13. The process of claim 1, whereby clusters of DNA matches involving more than three individuals may be evaluated by the analysis of three-element subsets of that collection.

14. The process of claim 1, whereby the SMD Utility maintains an application log to facilitate the auditing of mutually associated three-element subsets of collections larger than three elements.

15. The process of claim 1, whereby SMD may be deployed in conjunction with the backend server reporting of Shared Matches using the dataset of a commercial provider of genealogical DNA testing.

16. The process of claim 1, whereby SMD may be utilized to explore the latent ancestral relations of a collection of individuals without any a priori family trees.

17. The process of claim 1, whereby SMD may be employed to validate, or “proofread” collections of DNA matches obtained as the product of other bioinformatic processes including, but not limited to CMA (USPTO application Ser. No. #17/470,321).

18. The process of claim 1, whereby SMD may be employed as a pre-process to validate, or “proofread” collections of DNA matches to be used as input for other bioinformatic processes including, but not limited to AASK (USPTO application Ser. No. #18/641,045).
